(19) United States
(12) Patent Application Publication
(10) Pub. No.: US 2009/0312645 A1
(43) Pub. Date: Dec. 17, 2009

(54) Methods and Devices for Accessing Anatomic Structures

(75) Inventors: Barry Weitzner, Acton, MA (US); Christopher Thompson, Needham, MA (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Boston Scientific Scimed, Inc.

(21) Appl. No.: 12/482,081

(22) Filed: Jun. 10, 2009

Related U.S. Application Data
(60) Provisional application No. 61/129,268, filed on Jun. 16, 2008.

(51) Int. Cl. A61B 1/018; A61B 6/00

(52) U.S. Cl. 600/476; 600/106

(57) ABSTRACT

Described herein are methods and devices for performing endoscopic retrograde cholangiopancreatography (ERCP). A system comprising an elongate body housing a front-viewing endoscope and a tissue grasping instrument can be used to facilitate the insertion of a catheter into the ampulla of Vater. The distal end of the elongate body can be positioned such that the operator of the system can view the ampulla while manipulating the surrounding tissue to ease cannulation of the opening with the catheter. As a result, the procedure can be performed faster and more reliably than in the past.

### INCORPORATION BY REFERENCE

This application claims benefit of priority of U.S. Provisional Application No. 61/129,268, filed Jun. 16, 2008, the entire contents of which are incorporated herein by reference. This application also incorporates by reference the systems and methods described in Weitzner et al., U.S. patent application Ser. No. 11/946,779.

### BACKGROUND OF THE INVENTION

Endoscopic retrograde cholangiopancreatography (ERCP) is a technique that combines the use of endoscopy and fluoroscopy to diagnose and treat certain problems of the biliary or pancreatic ductal systems. It is an x-ray examination of the bile ducts which is aided by a video endoscope. Through the endoscope, the physician can see the inside of the stomach and duodenum, and inject dyes into the ducts in the biliary tree and pancreas so they can be seen on x-rays.

During ERCP, the patient is often sedated or anaesthetized. Next, an endoscope is inserted through the mouth, down the esophagus, into the stomach, through the pylorus into the duodenum, to the ampulla of Vater (the opening of the common bile duct and pancreatic duct). Due to the shape of the ampulla and the angle at which the common bile and pancreatic ducts meet the wall of the duodenum, the distal end of the endoscope is generally placed just past the ampulla. Due to the positioning of the endoscope beyond the ampulla, the endscopes used in these procedures is usually side-viewing. The side-viewing feature provides imaging along the lateral aspect of the tip rather than from the end of the endoscope. This allows the endoscopist to obtain an image of the medial wall of the duodenum, where the ampulla of Vater is located, even though the distal tip of the endoscope is beyond the opening.

Next, a user cannulates the entrance to the pancreatic and bile ducts, which are located beyond the ampulla of Vater, with a catheter or cannula placed through the instrument channel of the endoscope. The catheters are directed cranially at an angle with respect to the distal end of the endoscope, so as to facilitate insertion into the opening. Once in place within the ampulla, a radiocontrast agent can be injected into the bile ducts and/or pancreatic duct. Fluoroscopy can then be used to identify and treat various ailments, including blockages or leakage of bile into the peritoneum (abdominal cavity).

### SUMMARY OF THE INVENTION

Described herein are systems and methods for performing endoscopic retrograde cholangiopancreatography. In one aspect, the system comprises an elongate body having a plurality of instruments, including a tissue manipulation tool, a catheter tool, and an optical device. The instruments can be controlled via user inputs located at the proximal end of the elongate body.

In one aspect, the elongate body is sized and shaped so as to access the ampulla of Vater in the lateral wall of the duodenum via the patient's esophagus and stomach.

In another aspect, the tissue manipulation tool, the catheter tool, and the optical device comprise elongate shafts at least partially housed within the elongate body of the system coupling the working or distal ends of these instruments to user controls on the proximal ends of the shafts.

In another aspect, the distal end of the elongate body of the system can be positioned proximate to the ampulla of Vater where the tissue manipulation tool can manipulate the tissue surrounding the opening so as to facilitate easier insertion of the distal end of the catheter tool into the ampulla while the optical device is positioned such that the operator of the system can visualize the procedure.

It is to be understood that both the foregoing general description and the following detailed description are exemplary and explanatory only and are not restrictive of the invention, as claimed.

The accompanying drawings, which are incorporated in and constitute a part of this specification, illustrate several embodiments of the invention and together with the description, serve to explain the principles of the invention.

### BRIEF DESCRIPTION OF THE DRAWINGS

FIG. 1 is a cross-sectional view of the human body.

FIG. 2 is a perspective view of a portion of the digestive tract.

FIG. 3 is a side view of one embodiment of a device disclosed herein.

FIG. 4A is a cross-sectional front view of one embodiment of a device disclosed herein.

FIG. 4B is a cross-sectional front view of one embodiment of a device disclosed herein.

FIG. 5 is a cross-sectional side view of one embodiment of a device disclosed herein.

FIG. 6 is a side view of one embodiment of a device disclosed herein.

FIG. 7A is a cross-sectional side view of one embodiment of a device disclosed herein.

FIG. 7B is a cross-sectional side view of one embodiment of a device disclosed herein.

FIG. 7C is a cross-sectional side view of one embodiment of a device disclosed herein.

FIG. 7D is a side view of one embodiment of a device disclosed herein.

FIG. 8 is a side view of one embodiment of a device disclosed herein.

FIG. 9A is a side view of one embodiment of a device disclosed herein.

FIG. 9B is a side view of one embodiment of a device disclosed herein.

FIG. 10 is a side view of one embodiment of a device disclosed herein.

FIG. 11 is a side view of one embodiment of a device disclosed herein.

FIG. 12 is a side view of one embodiment of a device disclosed herein.

FIG. 13 is a side view of one embodiment of a device disclosed herein.

### DESCRIPTION OF EXEMPLARY EMBODIMENTS

Disclosed herein are systems and methods for cannulating the ampulla of Vater during the diagnosis and treatment of biliary, hepatic, gallbladder, and/or pancreatic disease or other ailments.

Generally, conventional systems allow an operator to navigate the distal end of an instrument-carrying endoscope from the mouth of a patient, down the esophagus, and through the stomach to the patient's duodenum, where the ampulla of Vater is located. Due to the shape of the ampulla and the angle at which the opening meets the wall of the duodenum, physicians performing this procedure typically position the distal end of the endoscope beyond the ampulla and then cannulate the opening by feeding the catheter back, cranially, towards the ampulla. In order to visualize the ampulla from a position beyond it, a side-viewing endoscope, as opposed to a more intuitive and easier to operate front-viewing endoscope, is used. Once the endoscope is properly positioned, the physician can attempt to guide the distal tip of the catheter into the ampulla.

Applicants have found that the side-viewing feature of current endoscopes may be more difficult to operate. Additionally, the limited manuverability of conventional tools can result in failed attempts at cannulation and the associated swelling and irritation of the ampulla of Vater. As a result, not only is the traditional procedure challenging, but the failure to properly place the catheter tip within the ampulla on one of the first two or three tries can greatly reduce the chances of successfully completing the procedure.

The devices and methods disclosed below are designed to solve these problems with the inclusion of a tissue manipulating tool located at the distal end of the endoscope, proximate to the distal end of the catheter. In one aspect, the tissue manipulating tool can facilitate easier insertion of the catheter into the ampulla by manipulating the tissue located adjacent the opening and making the bile and pancreatic ducts more accessible. In another aspect, the tissue manipulating tool can be used to manipulate the tissue adjacent the ampulla in such a way that the opening can be visualized without positioning the distal end of the endoscope beyond the ampulla. Thus, a front-viewing endoscope, rather than a more difficult to operate side-viewing endoscope, can be used during the procedure to aid the operator in cannulating the opening. These features can result in faster, more reliable cannulation of the bile and/or pancreatic ducts.

While the systems and methods described herein focus on cannulation of the ampulla of Vater, one skilled in the art will appreciate that the devices, systems, and methods of use described below can permit cannulation of a variety of anatomic structures. In one aspect, the system is sized and shaped for trans-oral access to the duodenum. However, in other embodiments, the system can be designed to access anatomic structures via other openings in the body. In another aspect, the system is configured specifically for cannulation of the ampulla of Vater. But the methods and devices described herein can be used for other, non-biliary or non-pancreatic procedures including, but not limited to, treatment of diverticulitis, drainage of cysts, or other gastrointestinal tract ailments. Additionally, these methods and devices are not limited to use in human patients. They can be performed and used in animals as well.

Reference will now be made in detail to the exemplary embodiments of the invention, examples of which are illustrated in the accompanying drawings. Wherever possible, the same reference numbers will be used throughout the drawings to refer to the same or like parts.

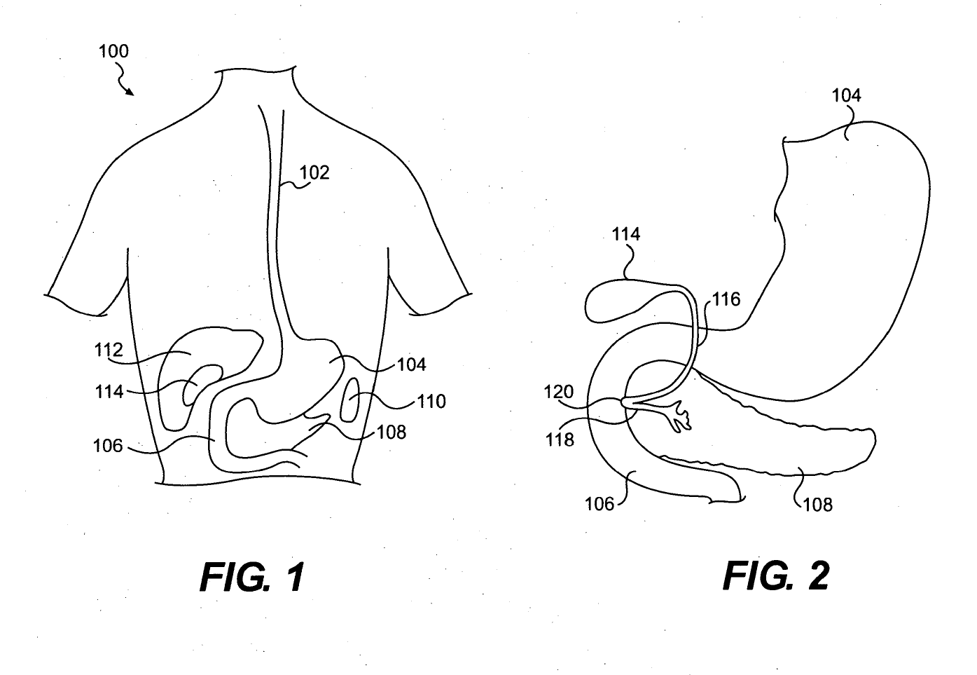

FIG. 1 depicts a portion of the anatomy of a patient 100. Beginning at the throat, the esophagus 102 leads down to the stomach 104. The stomach then leads to the duodenum 106, the upper portion of the small intestines. Surrounding the stomach and duodenum are the pancreas 108, the spleen 110, the liver 112, and the gallbladder 114.

In one aspect of the methods described herein, the physician can access the duodenum by inserting a guide tube into the mouth of the patient and guiding it down esophagus 102, through stomach 104, and into the upper intestines.

FIG. 2 illustrates duodenum 106 in more detail. Along the lateral wall of the duodenum is the ampulla of Vater 120. Through this opening lies the common bile duct 116, which leads to gallbladder 114, and the pancreatic duct 118, which leads to pancreas 108.

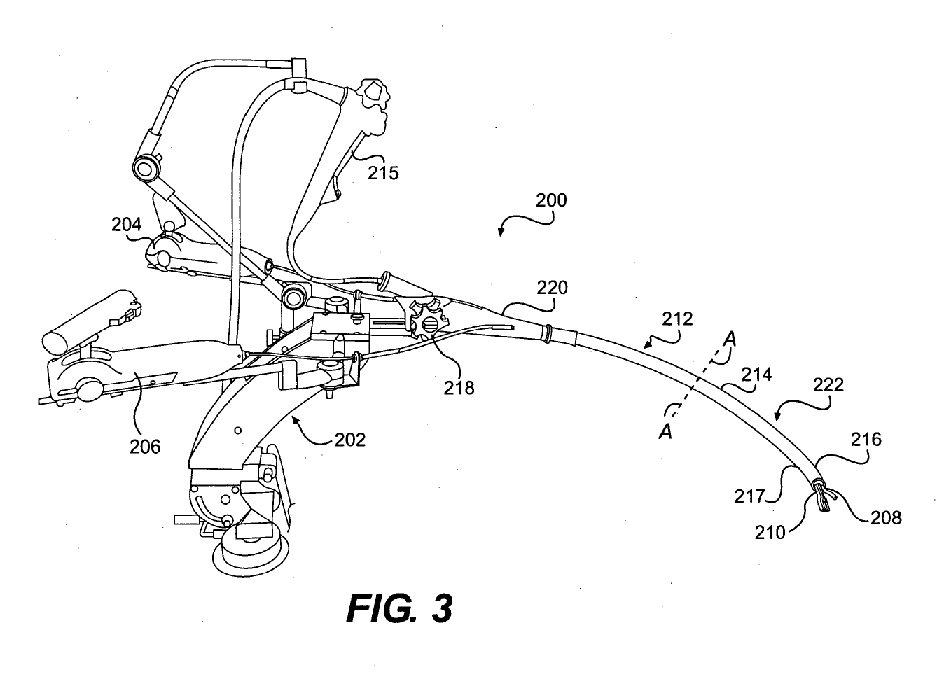

FIG. 3 provides a perspective view of one embodiment of a system 200 for performing ERCP and/or cannulating ampulla of Vater 120 according to the methods described herein. It should be noted, however, other devices and/or endoscopes can be used for this procedure and FIG. 3 depicts just one example of such a device.

The system includes a frame 202 for supporting control members 204 and 206 of tools 208 and 210, respectively, and a guide tube 212 for housing the elongate body of tools 208 and 210 and/or an optical device 215. When guide tube 212 is inserted into a patient, control members 204 and 206 allow a surgeon to manipulate surgical tools 208 and 210 which each have multiple degrees of freedom and extend to a surgical site positioned adjacent to a distal end 216 of guide tube 212. Frame 202 can have a variety of configurations depending on patient location, spacing, ergonomics, physician preference, and/or the availability of an operating table frame.

Guide tube 212 can have an elongate body 214 extending from the frame and configured for insertion through the mouth to a surgical site within a patient. In other embodiments, however, guide tube 212 can be configured for insertion in some other natural orifice or through an incision in the patient. While the guide tube is shown in FIG. 3 as mated with frame 202, guide tube 212 can be used without frame 202 during a portion or all of a surgical procedure. In one aspect, guide tube 212 includes a distal articulating end 216 that is controlled by proximal guide tube controls 218. A proximal end 220 of the guide tube can include at least one aperture for receipt of surgical instruments, such as, for example, tools 208, 210, and/or optical device 215 (together generally referred to herein as “surgical instruments”). Between proximal end 220 and distal end 216 of guide tube 212, elongate body 214 can include a mid-portion 222. In one embodiment, mid-portion 222 is generally flexible and non-articulating. In another embodiment, at least a portion of the guide tube is rigid. For example, a portion or the whole of guide tube 212 can be rigid.

In one embodiment, as discussed below, guide tube 212 can provide system 200 with one, two, or more than two degrees of freedom. For example, guide tube 212 can be articulated with controls 218 to move at least a portion of guide tube 212 (e.g., distal end 216) up/down and/or side-to-side. Additional degrees of freedom, provided for example, via rotation, translational movement of the guide tube with respect to the frame, patient, and/or point of reference, and/or additional articulation or bending sections, are also contemplated.

The outer surface of elongate body 214 of guide tube 212 can include a layer of lubricous material to facilitate insertion of guide tube 212 through a body lumen or surgical insertion. The interior of elongate body 214 can include at least one channel adapted to guide at least one elongate surgical instrument to a surgical site. In another aspect, the body can have two channels, three channels, or more than three channels. In one aspect, the guide tube includes multiple channels comprising a main channel for receipt of an optical device, such as an endoscope, and working channels for receipt of articulating surgical tools. The number of channels and their particular configuration can be varied depending on the intended use of the system and the resultant number and type of surgical instruments required during a procedure. For example, the guide tube can include a single channel adapted to receive multiple instruments or multiple channels for multiple instruments.

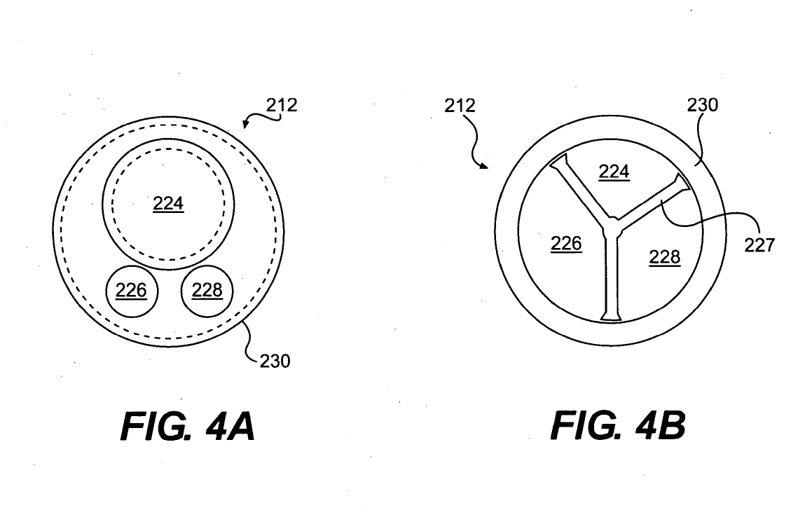

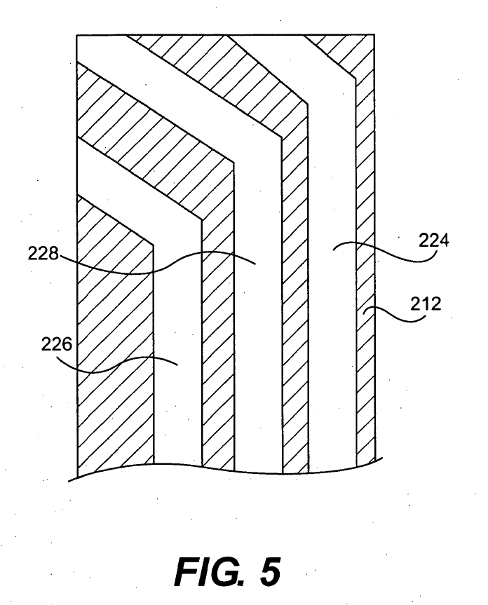

FIGS. 4A and 4B illustrate exemplary cross-sectional views of the mid-portion of elongate body 214 (taken along line A-A in FIG. 3) that includes main channel 224 and working channels 226 and 228. While three channels are illustrated, fewer channels (e.g., one or two) or more channels (e.g., four or more) are also contemplated. In addition, while main channel 224 is described as the largest channel, in terms of cross-sectional width, the working channels 226 and 228 can be a larger or smaller size than main channel 224. Moreover, use of the word “channel” does not require that the optical devices and/or surgical instruments traversing the guide tube be distinct or stand alone devices. For example, in one embodiment, the system includes an optical device and/or surgical instrument formed integrally with the guide tube. In still another embodiment, the optical devices and/or instruments described herein can, themselves, define the guide tube. For example, the optical device can define the guide tube and include channels for instruments. Channels may also be located on the outside of guide tube 212.

Regardless, in the exemplary illustrated embodiment of FIG. 4A, main channel 224 can be defined by at least one elongate lumen that extends, at least partially, between proximal end 220 and distal end 216 of guide tube 212. Similarly, working channels 226 and 228 can be defined by separate lumens, with main and working channels housed in an outer lumen. Alternatively, as illustrated in FIG. 4B, at least one of channels 224, 226, and 228 can be defined by a divider 227 that extends along at least a portion of guide tube 212. For example, all three channels 224, 226, and 228 can share a common sheath or outer jacket 230. One skilled in the art will appreciate that divider 227 can be defined by a portion of the guide tube and/or by a separate element that is mated with the guide tube and/or instruments.

Additional details concerning apparatus 200, including the construction and features of main channel 224 and working channels 226 and 228 can be found in U.S. patent application Ser. No. 11/946,779, incorporated herein by reference.

Referring again to FIG. 3, distal to the mid-portion 222 of elongate body 214, the guide tube can include an articulation portion 217. In one aspect, the articulation portion provides at least one degree of freedom, and in another aspect, provides more than one degree of freedom (e.g., two, three, or more than three degrees of freedom) to system 200. In particular, the distal end of the guide tube can be moved side-to-side and/or up/down by proximal controls 218. In another aspect, the guide tube can additionally, or alternatively, move longitudinally and/or rotate. Articulation, regardless of the number of degrees of freedom, can be controlled in a variety ways.

In one aspect, the main channel is adapted to articulate while the working channels are mated to the main channel and move with the main channel. In other words, the working channels are not directly articulated. However, in another aspect, all the channels can be directly articulated together or independently depending on the intended use of system 200. Another embodiment includes a single lumen that articulates and is configured to receive multiple instruments or multiple channel bodies. For example, the guide tube can include one working channel for receiving multiple instruments.

A variety of mechanisms can be used to manipulate articulation portion 217 and/or articulation portions of tools 208 and 210 or optical device 215, including, for example, push-pull strands, leaf springs, cables, oversheaths, ribbons, electroactive materials, prebent materials, and/or fluid actuation. Examples of these mechanisms and additional methods of articulating both the guide tube and the instruments within the tube are provided in U.S. patent application Ser. No. 11/946,779. Other mechanisms known in the art can also be incorporated.

In regards to articulating tools 208, 210, or optical device 215, in addition to the articulation mechanisms mentioned above, channels 224, 226, and 228 can be shaped and directed so as to articulate the instruments. FIG. 5 depicts such a configuration. In one aspect, the working and/or main channels have an angled configuration relative to the longitudinal axis of guide tube 212 such that the surgical instruments are directed to one side as they exit the guide tube proximate to the tube's distal end. They may also be directed away from each other to wrap around the work space. This is referred to as triangulation. In other embodiments, the channels can be retractable to further guide the instruments.

As tools 208 and 210 and/or optical device 215 are fed through channels 224, 226, and 228 and reach the distal end thereof, they follow the path of the channels and exit guide tube 212 at generally the same angle as the channels with respect to the tube. For example, one of the channels can exit the guide tube at about a 30° angle with respect to the longitudinal axis of the tube. As a result, a tool advanced through that channel can also exit guide tube 212 at about a 30° angle. In other embodiments, this angle may be greater or less than 30°. In different embodiments, only one of the channels can be angled while the remaining channels run parallel to the longitudinal axis of guide tube 212 and exit its distal end. In still other embodiments, more than one or all of the channels can exit the guide tube at some angle. Further, the angle at which each channel exits the guide tube need not be the same. Additionally, not only can channels such as those just described articulate the instruments exiting the guide tube in a predetermined manner, but they can also serve to maintain some predetermined spacing between the distal ends of the tools while the system is in use. For example, working channels 224 and 226 can exit the distal end of guide tube 212 some distance apart and/or at diverging angles to ensure that the tools placed within these channels are adequately spaced within the working area of the patient. The channels can also be individually advanceable or otherwise moveable.

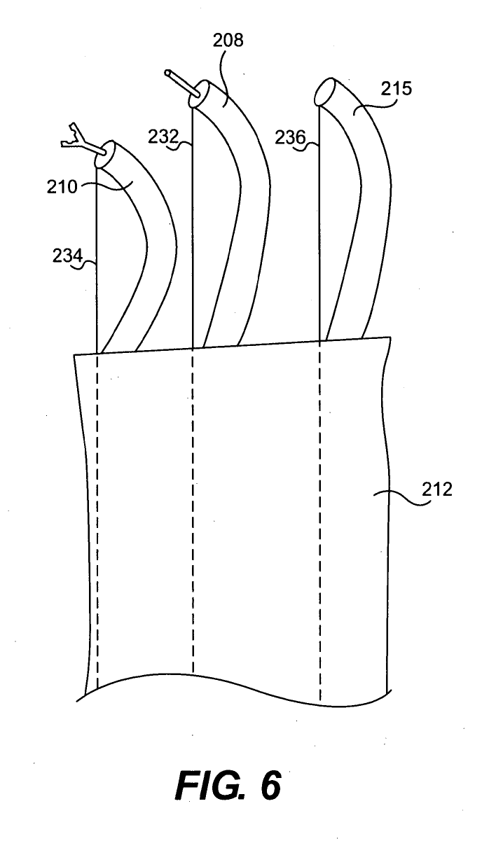

Instead of, or in addition to angled lumens, the guide tube can include an active lumen that can be articulated. In one aspect the guide tube channels can be articulated. For example, FIG. 6 illustrates filaments or control wires 232, 234, and 236, mated with the guide tube channels, such that tensioning the wires causes the channels to bend and articulate the instruments. The channels, in one aspect, also include a bias for bending in one direction. In another embodiment, the channels can include a pre-bend or shape memory material that moves into a bent position when unconstrained by the guide tube and/or after exposure of channels 224, 226, and/or 228 to a trigger (e.g., body heat). Furthermore, pneumatics or hydraulics can be employed to articulate the channels.

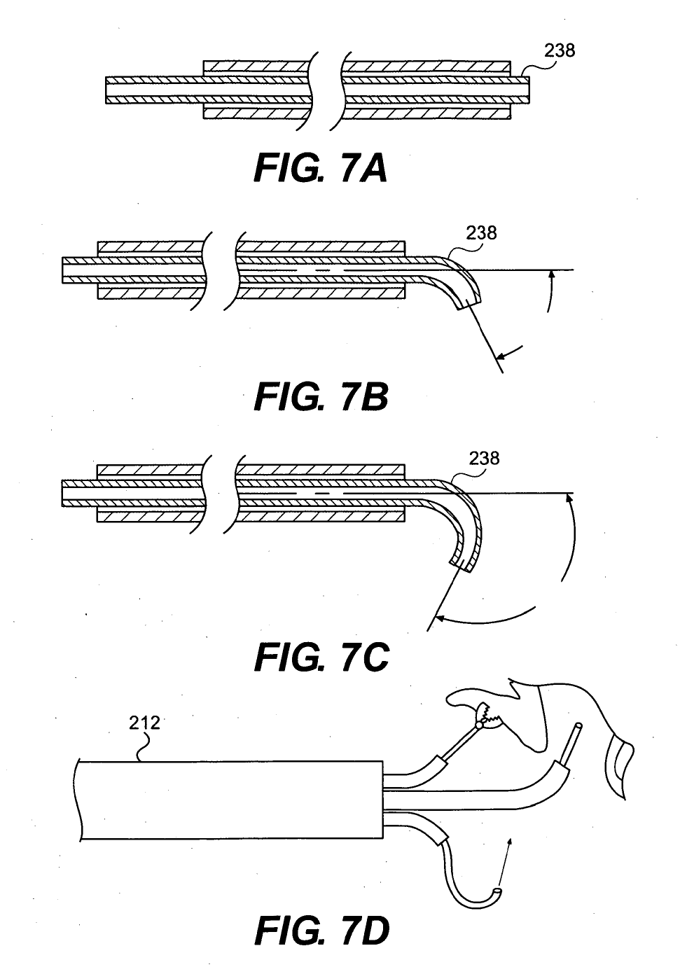

In another embodiment described herein, one or more of the channels within guide tube 212 (e.g., channel 224) can allow increased curvature or retro-flexing. As illustrated in FIGS. 7A through 7D, a channel of guide tube 212 can include telescoping curved body 238 that when extended from the distal end of the channel, assumes a curvature. In one aspect, the curvature can comprise at least 45°, in another aspect, a curve of at least at 90°, and in yet another aspect, a curve of at least 150° or more. The curved body (or bodies) can thus provide one or more than one additional degrees of freedom to the system.

In another embodiment, an s-curve is provided. For example, body 238 can include a first and a second pre-formed curve that bend in opposite directions. In another aspect, body 238 provides a first curve and a controllable instrument is extended through body 238 and bent to provide a second curved portion.

The curved bodies can have a pre-formed curvature that is constrained by a portion of system 200. In one aspect, a channel of guide tube 212 constrains curved body 238. A user can push body 238 out of the end of the channel, allowing body 238 to bend with respect to the channel. In another aspect, a stiffening member can constrain the curve body. Withdrawing the stiffening member can allow the channel and/or surgical instrument to bend into a pre-curved configuration. Alternatively, the stiffening member can be curved such that the channel can be curved by advancing the stiffening member therein or straightened by withdrawing the stiffening member therefrom.

In one aspect, body 238 can rotate in addition to translating with respect to guide tube 212 and/or the channel within which it is at least partially housed. In use, body 238 can be rotated relative to the channel to direct a surgical instrument in a desired direction. In one aspect, body 238 is rotated into the desired orientation prior to insertion of guide tube 212 into a patient. In another aspect, rotation of body 238 can be controlled by a user from a proximal location.

It should be noted, while specific examples of tool and/or optical device articulation have been set forth above, other methods exist and those mentioned herein should not be considered limiting. For example, various other methods for articulating tools 208 and 210 and optical device 215 are disclosed in U.S. patent application Ser. No. 11/946,779, incorporated herein by reference. Additionally, other methods are known in the art.

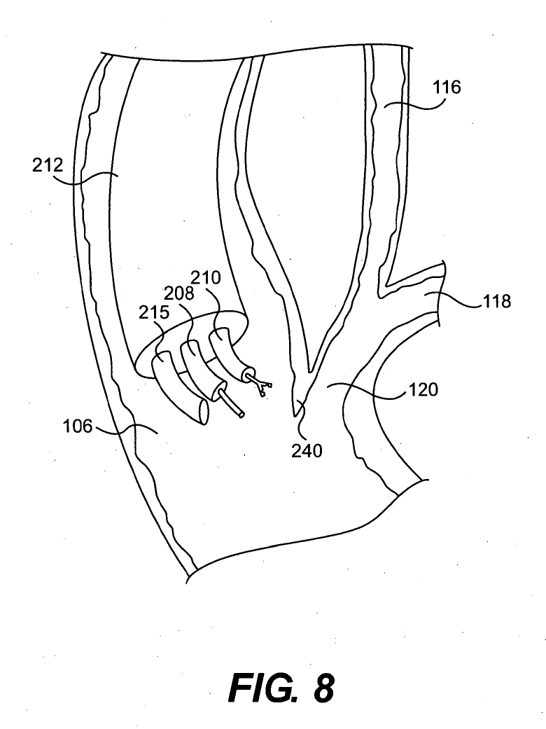

Referring now to FIG. 8, one embodiment of the device described above is depicted. In this embodiment, tool 208 can comprise an elongate catheter, the longitudinal axis of which can run parallel to the longitudinal axis of guide tube 212. Tool 210 can also include an elongate catheter body with an end effector, which in this case, is a pair of jaws. Optical device 215, in this embodiment, is an end-viewing endoscope, but, in other embodiments, the device can be a side-viewing scope or optical wand which may or may not be integral to the guide tube. In one aspect, one or more of these instruments can be comprised of an elongate shaft that runs through guide tube 212 for a portion or all of the tube's length. In another aspect, the distal end of the catheter, the jaws, and optical device 215 can all have multiple degrees of freedom, including, but not limited to, being rotatable, translatable, and capable of articulation in two degrees of freedom (left/right, up/down) with respect to the guide tube. Additional degrees of freedom, via, for example, movement of the guide tube with respect to a point of reference (e.g., the patient's anatomy) are also contemplated.

In practice, guide tube 212 can be positioned proximate to ampulla of Vater 120, on the cranial side of the opening. From this location, optical device 215 can be positioned and articulated so as to obtain a bird's eye view of a flap of tissue 240 partially obstructing the ampulla. Next, using proximal control member 206 (FIG. 3), grasping instrument 210 can be positioned proximate flap 240 and articulated such that the instrument can be actuated, again from proximal control member 206, so as to grasp flap 240. In another embodiment, optical device 215 can be situated coaxially within tool 208 or 210. Thus, no separate lumen within the guide tube is needed to house the optical device. Or the guide tube can comprise two or more optical devices to gain more than one perspective on the opening to be cannulated.

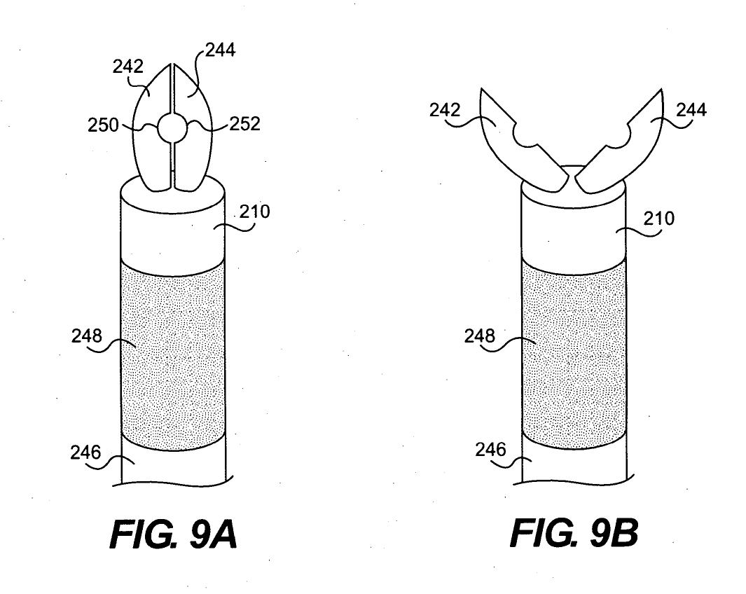

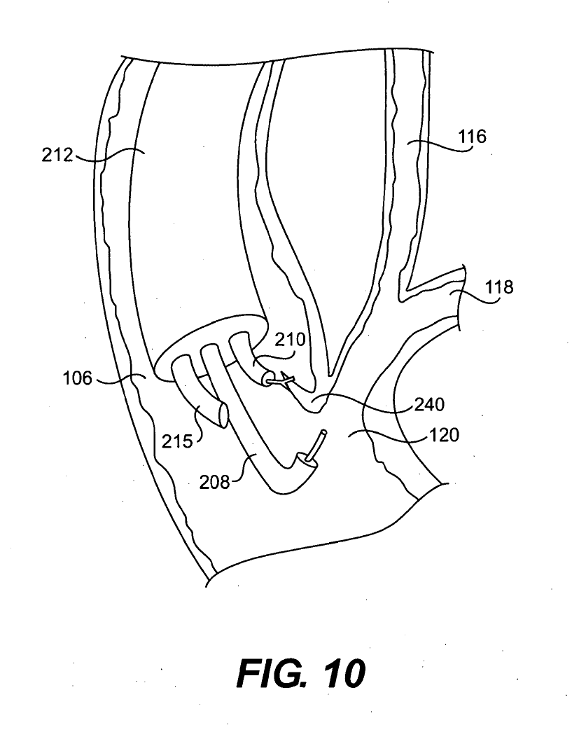

FIGS. 9A and 9B provide a more detailed view of one embodiment of grasping instrument 210. In one aspect, a shaft 246 of tool 210 can comprise an articulating segment 248. In another aspect, the instrument can include a pair of jaws, 242 and 244, hingedly affixed to the distal end of shaft 246. In some embodiments, shaft 246 runs the entire length of guide tube 212. In other embodiments, the shaft runs only a portion of the length of the guide tube. In another aspect, the actuation of the jaws (the grasping action), as well as the articulation and additional degrees of freedom of shaft 246 can be controlled from a single control member 206.

In some embodiments of the device, jaws 242 and 244 can have corresponding recesses 250 and 252, respectively, in their opposing grasping surfaces. This recess can be used to grasp, without damaging, other objects, such as tissue or another instrument. For example, this recess can be used to grasp the body of another tool such as the cannulating instrument and/or optical device. Movement of tool 210 can thus be used to articulate tool 208 via an articulation segment of tool 210 and/or via longitudinal and/or rotational movement of tool 210.

Referring now to FIG. 10, once tissue flap 240 has been secured by grasping instrument 210, instrument 210 can be repositioned cranially or in some other direction with respect to ampulla 120 so as to displace flap 240 away from the ampulla and at least partially unobstruct the opening. In addition to repositioning tool 210, the grasping instrument can also be rotated or articulated in order to adequately displace flap 240 from opening 120. As a result, optical device 215 can a better, e.g, less obstructed, view of ampulla 120 and the opening can be more accessible to catheter tool 208. Next, the distal end of the catheter can be moved longitudinally, rotated, and/or articulated so as to align catheter tool 208 and opening 120. Finally, catheter tool 208 can be moved into the ampulla to achieve cannulation.

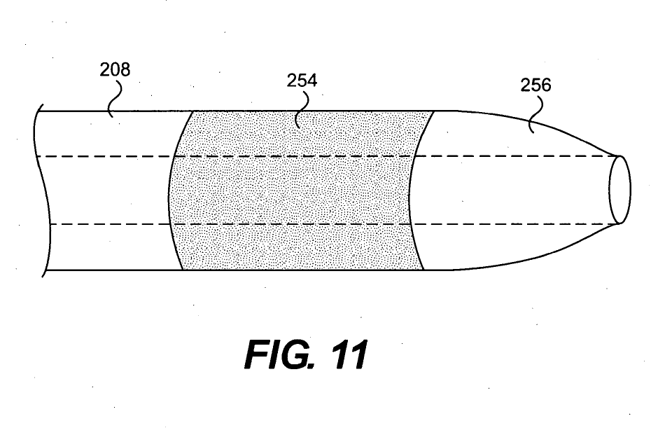

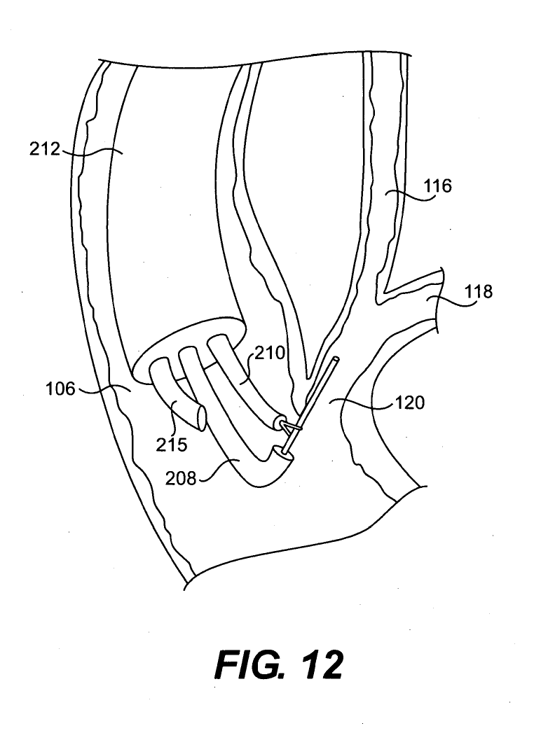

FIG. 11 depicts one embodiment of the distal tip of catheter 208. In one aspect the catheter has an articulating portion 254 similar to that exhibited by tool 210. In another aspect, the catheter can exhibit a tapered tip 256.

Once cannulated, catheter 208 can be guided, using control member 204, into either common bile duct 116 or pancreatic duct 118. Next, the physician can inject the desired duct with a radiopaque dye, or some other traceable or otherwise visible fluid, through catheter 208. By monitoring the injected dye, via x-ray, MRI, ultrasound, optically, etc., the physician can diagnose the patient's ailment(s) and set a course for future treatment, or immediate treatment if desired.

While tool 210 has been described above as comprising a pair of jaws, a variety of other end effectors can be used with tool 210. In an alternative embodiment, depicted in FIG. 13, tool 210 can deliver suction. The distal end of tool 210 can be pressed against the tissue to be manipulated (e.g., flap 240) and a vacuum delivered through the tool. The suction tool 210 can manipulate tissue proximate to the opening to be cannulated as described above. For example, tissue can be manipulated by driving a degree of freedom of tool 210 (longitudinal movement, rotational movement, articulation of the articulation segment) via control member 206. Alternatively, suction tool 210 and catheter 208 can be combined into one instrument. For example, catheter 208 can comprise a suction hood coaxial to the catheter's distal end.

Other embodiments of tool 210 are also possible and the list of embodiments recited herein should not be construed as exhaustive. For example, tool 210 could comprise a shaft with a blunt tip for manipulating and repositioning targeted tissue. Or it could comprise a hook, barb, or needle for securing targeted tissue for manipulation. Other methods of tissue manipulation are known in the art as well as described in U.S. patent application Ser. No. 11/946,779, incorporated herein by reference.

As described above, tools 208 and 210 can have various degrees of freedom including, for example, rotational movement, longitudinal movement, articulation (up/down and/or left/right), and/or actuation (e.g., grasping with jaws). In another embodiment, the distal end of tools 208 and/or 210 can be moved laterally or transversely with respect to the elongate guide tube and/or with respect to a portion of the body of tools 208 and/or 210. For example, referring now to FIG. 10, the distal end of tool 208 has been moved longitudinally in a distal direction from the distal end of guide tube 212 and articulated toward the ampulla of Vater. The distal end of tool 208 is now generally aligned with opening 120. In order to cannulate ampulla 120, the distal end of tool 208 can be moved transversely, with respect to guide tube 212, toward opening 120.

In one embodiment, tool 208 is positioned within an articulating sleeve as described above with respect to FIGS. 6 through 7D. Sleeve 238 can articulate or bend toward the ampulla to direct tool 208 toward the ampulla. Longitudinal movement of tool 208 with respect to the sleeve and/or guide tube can move the distal end of tool 208 in a transverse direction toward the ampulla opening 120 to permit cannulation. In one aspect, tool 208 does not include an articulation section. Alternatively, tool 208 includes an articulation section (for example, as described with respect to FIG. 11) which allows further control of the alignment between opening 120 and tool 208.

Sleeve 238 in one aspect, can extend the majority or full length of guide tube 212 and/or tool 208. Alternatively, sleeve 238 can be mated with guide tube 212 proximate to the distal end of the guide tube. Bending of sleeve 238 can be achieved via an articulation section as described with respect to tools 208 and 210. For example, one or more pull wires can extend from a proximal controller to the sleeve articulation section. Alternatively, the sleeve can include a pre-bent material as described above. In yet another embodiment, the sleeve does not bend. Instead, the sleeve can be rigid or partially rigid with a bent or curved section that is not actuated. In use, the sleeve can be rotated into position to direct the one or more tools toward opening 120.

In another embodiment, tools 208 and/or 210 can include two or more articulation sections spaced longitudinally. In other words, tools 208 and/or 210 can include a “wrist” and “elbow”. Together, the articulation sections can move the distal end of tools 208 and/or 210 transversely toward opening 120 and permit cannulation.

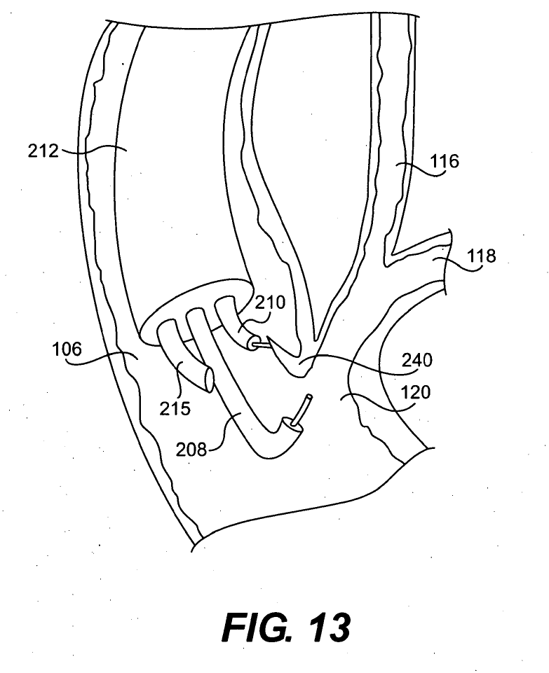

In still another embodiment, the tools (e.g., tools 208 and 210) can work together to move tool 208 into alignment with opening 120 and move tool 208 transversely. As mentioned above, during cannulation of the ampulla, the physician can also use grasping tool 210, via control member 206, to aid in guiding catheter tool 208 either into ampulla 120 or into either common bile duct 116 or pancreatic duct 118. FIG. 12 depicts such a step. Jaws 242 and 244 of tool 210 can grasp tool 208 and bend the body of tool 208 toward opening 120 and/or pull tool 208 transversely. Thus, the use of tools 208 and 210 together can provide additional control over the catheter tool 208.

In addition, or alternatively, tool 210 can grasp and manipulate tool 208 after cannulation or partial cannulation. Once the catheter is at least partially inserted into ampulla 120, the physician can release tissue flap 240 by opening jaws 242 and 244. The physician can then reposition grasping instrument 210 proximate to the shaft of catheter 208 such that the catheter body is between the two jaws. The jaws can then be actuated in order to clamp down on the catheter. In one aspect, tool 210 can be positioned around catheter 208 such that when the grasping instrument is actuated and jaws 242 and 244 converge on the catheter tool, the catheter tool body is secured in recesses 250 and 252 of the jaws. In one aspect, the recesses can facilitate gripping and/or reduce the chance of damaging the catheter by crushing it between jaws 242 and 244. Once tool 210 has a sufficient grasp on catheter 208, the grasping instrument can be repositioned, rotated, or articulated, via control member 206, in order to further assist catheter 208 into a desired position.

While the above discussion of transverse movement and manipulation of catheter tool 208 is focused on tools 208 and 210, it should be appreciated that more than two tools can be used and/or the various disclosed features can be applied to optical device 215.

Additional features can also be incorporated into the systems and methods described herein to improve its functionality. For example, the components of the device can be comprised of a medical grade material suitable for a surgical environment or a radiopaque material so as to permit visualization of the device during the procedure. In other embodiments, force, pressure, strain, and/or temperature sensors can be incorporated into the device providing the surgeon with information about conditions of interest within either the duodenum, the ampulla of Vater, the common bile duct, the pancreatic duct, the gallbladder, or the pancreas. Additionally, the guide tube and/or tools can be polymer or elastomer coated in order to improve grip or reduce friction while in use. Other materials can also be used as a coating.

The systems and methods described herein can also be used to cannulate openings in other areas of the body aside from the ampulla of Vater. In fact, the devices can be used in any procedure where an opening not easily visualized or accessed is to be cannulated or entered.

Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

What is claimed is:

1. A method for performing endoscopic retrograde cholangiopancreatography, comprising:
providing an endoscope having a proximal and distal end and an instrument channel, the instrument channel at least partially housing a catheter and an elongate tool, both the catheter and the elongate tool having a proximal and distal end;
positioning the distal end of the endoscope substantially proximate to an ampulla of Vater;
manipulating a portion of tissue adjacent the ampulla of Vater with the distal end of the elongate tool; and
cannulating the ampulla of Vater with the distal end of the catheter.

2. The method of claim 1, wherein the step of positioning the distal end of the endoscope proximate to the ampulla of Vater comprises translating, rotating, and/or articulating the endoscope.

3. The method of claim 1, wherein the step of positioning the distal end of the endoscope proximate to the ampulla of Vater comprises translating, rotating, and/or articulating the instrument channel.

4. The method of claim 1, wherein the distal end of the elongate tool comprises a pair of jaws and the step of manipulating the portion of tissue adjacent the ampulla of Vater comprises grasping the portion of tissue between the jaws.

5. The method of claim 1, wherein the step of positioning the distal end of the endoscope proximate to the ampulla of Vater comprises translating, rotating, and or articulating the elongate tool.

6. The method of claim 1, wherein the step of cannulating the ampulla of Vater with the distal end of the catheter comprises translating, rotating, and/or articulating the catheter.

7. The method of claim 1, wherein the step of manipulating the portion of tissue adjacent the ampulla of Vater comprises grasping the tissue and translating, rotating, pushing, and or pulling the portion of tissue.

8. The method of claim 1, wherein the step of cannulating the ampulla of Vater comprises grasping the catheter with the elongate tool and translating, rotating, and/or articulating the elongate tool.

9. The method of claim 1, further comprising the step of injecting a dye through the distal end of the catheter.

10. The method of claim 8, wherein the dye is a radiopaque dye.

11. The method of claim 1, further comprising the step of cannulating the common bile duct.

12. The method of claim 1, further comprising the step of cannulating the pancreatic duct.

13. A method of diagnosing gastrointestinal ailments and/or pancreatic disease or ailments, comprising:
providing an elongate instrument having a proximal and distal end, the elongate instrument at least partially housing a tissue manipulating tool and a cannula;
inserting the elongate instrument into a mouth of a patient and advancing it down an esophagus, through a stomach, and at least partially into a duodenum;
positioning the distal end of the elongate instrument proximate to an opening in a wall of the duodenum;
manipulating a portion of tissue adjacent the opening in the wall of the duodenum with the tissue manipulating tool; and
cannulating the opening in the wall of the duodenum with the cannula.

14. The method of claim 13, wherein the elongate instrument further comprises at least one instrument channel at least partially housing either the tissue manipulating tool, the cannula, or both.

15. The method of claim 14, wherein the instrument channel can be translated, rotated, and/or articulated with respect to the elongate instrument.

16. The method of claim 14, wherein the instrument channel is integral with the elongate instrument.

17. The method of claim 16, wherein the instrument channel exits the distal end of the elongate instrument at an angle with respect to the longitudinal axis of the elongate instrument.

18. The method of claim 15, wherein the instrument channel, when unrestricted by the elongate instrument, can assume a bend of some angle with respect to the longitudinal axis of the distal end of the elongate instrument.

19. The method of claim 14, wherein the elongate instrument is further comprised of a filament having a proximal and distal end, the proximal end of the filament extending to the proximal end of the elongate instrument and the distal end of the filament being coupled to the distal end of the instrument channel.

20. The method of claim 19, wherein the instrument channel can be translated, rotated, and/or articulated by pulling or pushing on the filament.

21. The method of claim 20, wherein the instrument channel is biased to bend in a particular direction and/or at a particular angle when a force is applied to the filament.

22. A device for cannulating an anatomic opening, comprising:
an elongate body having a proximal user input end and a distal manipulation end and an instrument channel therebetween;
a tissue manipulation tool at least partially housed within the instrument channel, the tissue manipulation tool having a proximal end and a distal end, the distal end being proximate to the manipulation end of the elongate body;
a cannula at least partially housed within the instrument channel, the cannula having a proximal end and a distal end, the distal end being proximate to the manipulation end of the elongate body; and
a controller for receiving user input and mechanically transmitting those user inputs to the distal end of the tissue manipulation tool and/or the cannula.

23. The device of claim 22, further comprising an optical device.

24. The device of claim 23, wherein the optical device is an end-viewing optical device.

25. The device of claim 22, wherein the distal end of the tissue manipulation tool comprises a pair of jaws.

26. The device of claim 25, wherein the jaws can be actuated from the controller.

27. The device of claim 25, wherein each jaw further comprises a recessed portion.

28. The device of claim 22, wherein the distal end of the tissue manipulation tool comprises a suction element.

29. The device of claim 22, wherein the distal end of the tissue manipulation tool comprises a hook.

30. The device of claim 22, wherein the distal end of the tissue manipulation tool comprises a needle.

31. The device of claim 22, wherein the tissue manipulation tool comprises an articulation portion.

32. The device of claim 22, wherein the cannula comprises an articulation portion.

33. The device of claim 22, wherein the distal end of the cannula comprises a tapered portion.
